(19) United States
(12) Patent Application Publication
(10) Pub. No.: US 2008/0033383 A1
(43) Pub. Date: Feb. 7, 2008

(54) Sanitary apparatus with moisture level indication

(76) Inventors: Joy Cantor, West Bloomfield, MI (US); Mark A. Cantor, West Bloomfield, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/497,766

(22) Filed: Aug. 2, 2006

(51) Int. Cl. A61F 13/42; G08B 21/20

(52) U.S. Cl. 604/361; 340/604

(57) ABSTRACT

A moisture absorbent sanitary apparatus is provided with a body, moisture absorbent material, a moisture sensor in the body, and an actuator in the body for imparting a motion to a user for indicating a level of moisture absorption. A computer-readable medium is disclosed with instructions for performing a method including receiving a signal of a moisture level in a sanitary apparatus, and transmitting a signal for driving an actuator in the sanitary apparatus.

### BACKGROUND OF THE INVENTION

1. Field of the Invention

The invention relates to sanitary apparatuses, more particularly sanitary apparatuses with moisture level indication.

2. Background Art

Sanitary apparatuses are utilized for absorbing moisture, such as that associated with menstruation, incontinence, child excrement or the like. Utilization of such sanitary apparatuses may lead to anxiety associated with the expectation of the sanitary apparatus reaching a full condition and subsequently leaking.

An aspect of the prior art is to provide a moisture level indicator with sanitary devices. Such sanitary devices often employ an external alert mechanism for alerting a user of a moisture level.

### SUMMARY OF THE INVENTION

In one non-limiting embodiment of the present invention, a moisture absorbent sanitary apparatus is provided having a body that is sized to cooperate with a body part of a user, with a moisture absorbent material for absorbing moisture from the user. A moisture sensor is provided in the moisture absorbent sanitary apparatus for sensing moisture at a location. An actuator is provided in the body of the apparatus in communication with the moisture sensor for receiving a signal from the moisture sensor indicating a level of moisture absorption in the moisture absorbent material whereby the actuator imparts a vibration, motion, or other signal to the body part of the user for indicating to the user that the moisture absorbent material has reached the level of moisture absorption.

Another non-limiting embodiment of the present invention provides a computer-readable medium having computer-executable instructions for performing a method for receiving a signal indicating a predefined moisture level in a moisture absorbent sanitary apparatus. The method includes transmitting a signal for driving an actuator in the moisture absorbent sanitary apparatus in response to receiving the signal for indicating to a user that the moisture absorbent sanitary apparatus has reached the predefined moisture level, such as a saturation level.

The above aspects, objects, embodiments, benefits and advantages are apparent in the attached figures and in the detailed description of embodiments of the invention below.

### BRIEF DESCRIPTION OF THE DRAWINGS

FIG. 1 is a perspective view of a sanitary apparatus in accordance with the present invention;

FIG. 2 is a perspective view of another sanitary apparatus in accordance with the present invention;

FIG. 3 is a perspective view of another sanitary apparatus in accordance with the present invention;

FIG. 4 is a schematic view of a moisture level indicator of a sanitary apparatus in accordance with the present invention;

FIG. 5 is a schematic view of another moisture level indicator of a sanitary apparatus in accordance with the present invention;

FIG. 6 is a perspective view of an actuator of a moisture level indicator of a sanitary apparatus of the present invention;

FIG. 7 is a schematic view of another actuator of a moisture level indicator of a sanitary apparatus of the present invention;

FIG. 8 is a partial section view of a moisture sensor of a moisture level indicator of a sanitary apparatus in accordance with the present invention;

FIG. 9 is a section view of a mode selection switch of a moisture level indicator of a sanitary apparatus in accordance with the present invention; and

FIG. 10 is a flowchart of a method performed by computer-executable instructions of a computer-readable medium of a moisture level indicator of a sanitary device in accordance with the present invention.

### DESCRIPTION OF EMBODIMENTS OF THE INVENTION

As required, detailed embodiments of the present invention are disclosed herein; however, it is to be understood that the disclosed embodiments are merely exemplary of the invention that may be embodied in various and alternative forms. The Figures are not necessarily to scale, some features may be exaggerated or minimized to show details of particular components. Therefore, specific structural and functional details disclosed herein are not to be interpreted as limiting, but merely as a representative basis for the claims and/or as a representative basis for teaching one skilled in the art to variously employ the present invention.

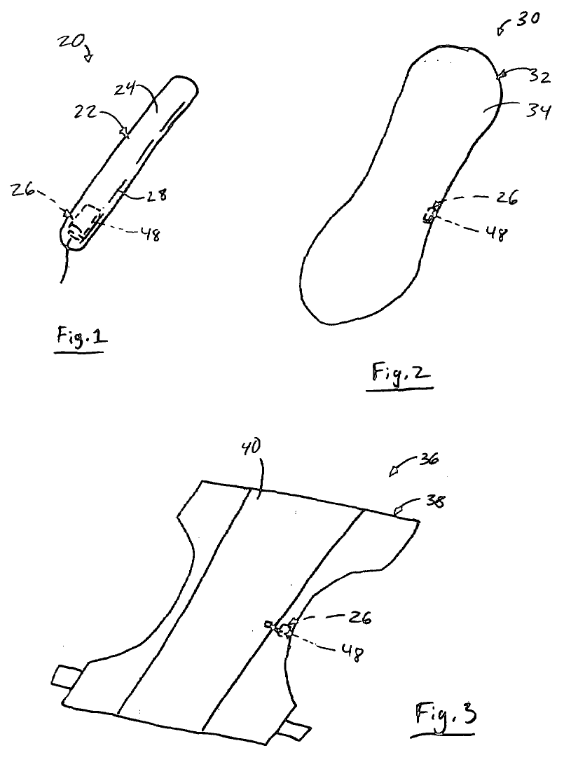

With reference now to FIG. 1, a moisture absorbent sanitary apparatus is illustrated, in accordance with the present invention, as a tampon and is referenced generally by numeral 20. The tampon 20 includes a body 22 that is sized to be inserted within a vaginal canal of a user for absorption of menstrual fluids. The body 22 includes moisture absorbent material 24 such as cotton, for absorbing the moisture conveyed to the tampon 20.

A moisture level indicator 26 is illustrated within the body 22 of the tampon 20. The moisture level indicator is provided within the body 22 at an orientation to sense moisture within the moisture absorbent material 24 and indicate the level of moisture to the user. Although the moisture level indicator 26 is illustrated at a distal end of the tampon 20 in FIG. 1 the invention contemplates that the moisture level indicator 26 may be provided at any orientation within the tampon 20 for providing adequate warning to the user of the moisture level of the tampon 20. The invention also contemplates utilization of various moisture level indicators for incrementally indicating to the user multiple moisture levels within the tampon 20.

The moisture level indicator 26 may be provided within the tampon 20 by the manufacturer, or may be installed by the user. The body 22 may include a series of slits 28 formed therein, each sized for receipt of the moisture level indicator 26. The user may select the desired level of moisture level indication by installing the moisture level indicator in the corresponding slit 28. Thus, the moisture level indicator 26 may be utilized for indicating to the user that the tampon 20 is full, almost full, or various levels of partially full. In one embodiment, the moisture level indicator 26 informs the user when the tampon 20 reaches saturation. Thus, accidents, such as leakage, may be prevented. Additionally, such indication will alert the user that a tampon 20 has already been inserted, thereby avoiding insertion of another tampon 20.

Although a tampon 20 is illustrated in FIG. 1, any sanitary apparatus is contemplated within the spirit and scope of the present invention.

Referring now to FIG. 2, another moisture absorbent sanitary apparatus is illustrated as a pad or napkin 30. The pad 30 has a body 32 that is sized to be worn by women to absorb menstrual flow. The body 32 includes a moisture absorbent material 34, such as cotton for absorbing fluids exuded from the user's body. The pad 30 also includes a moisture level indicator 26 within the body 32 for sensing a level of moisture absorption and indicating the user of the level.

Similar to the prior embodiment, the moisture level indicator 26 may be provided at any orientation or level of moisture absorption within the body 32 of the pad 30. Additionally, multiple moisture level indicators 26 may be provided within the body 32 of the pad 30.

With reference now to FIG. 3, another moisture absorbent sanitary apparatus is illustrated embodied as a diaper 36. The diaper 36 includes a body 38 that is sized to be worn by a user. Various sizes of the body 38 may be provided for various size users, such as various size children for absorbing and collecting child excrement, or for adults for absorbing and collecting adult excrement.

A moisture absorbent material 40 such as cotton, is provided in the body 38 for absorbing and collecting the excrement of the user. Similar to the prior embodiments, a moisture level indicator 26 is provided within the body for sensing when the moisture absorbent material 40 reaches a particular moisture absorption level. Likewise, the moisture level indicator 26 may be provided at various locations, the moisture level indicator 26 may be installed by the manufacturer or the user, and the body 38 of the diaper 36 may be provided with multiple level indicators 26.

The invention also contemplates that the moisture absorbent sanitary apparatus may be embodied in various forms such as those disclosed, and as a bandage for dressing a wound, or the like.

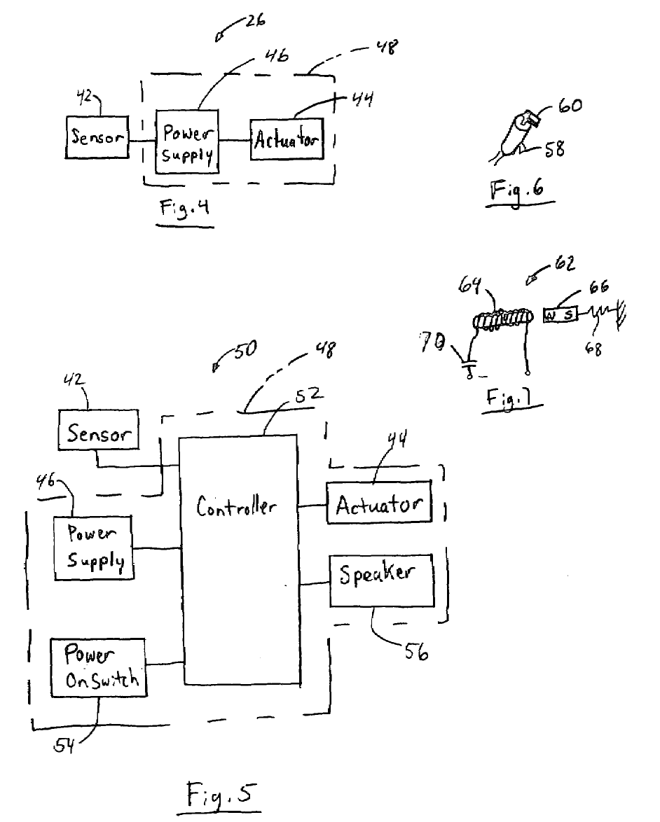

FIG. 4 illustrates the moisture level indicator 26 schematically. The moisture level indicator 26 includes a moisture sensor 42 for sensing moisture at the location at which the moisture level indicator 26 is provided. The sensor 42 communicates with an actuator 44 of the moisture level indicator 26. Upon receiving a signal from the sensor 42, the actuator 44 imparts a motion, vibration or other signal to the user to indicate that the moisture absorbent sanitary apparatus has reached the predefined moisture level.

The moisture level indicator 26 may also include a power supply 46 such as a battery for providing power to the actuator 44. The sensor 42, actuator 44 and power supply 46 may be in series such that the sensor 42 closes the circuit thereby conveying the power from the power supply 46 to the actuator 44.

In order to prevent contamination of the actuator 44 or power supply 46, the actuator 44 and power supply 46 may be provided within a cover 48. The cover 48 may be formed from a moisture resistant material such as a polymeric material like polyvinyl chloride (PVC). The cover 48 may be a cover that the power supply 46 and actuator 44 are assembled within. The cover 48 may be formed about these components, for example by injection molding. The cover 48 prevents moisture from breaching the actuator 44 and the power supply 46 and likewise conceals the actuator 44 and the power supply 46 for the safety of the user.

Referring now to FIG. 5, another moisture level indicator 50 is illustrated in accordance with the present invention. The moisture level indicator 50 includes a moisture sensor 42 and an actuator 44 in communication through a controller 52. The controller 52 may be a microprocessor, an integrated chip, or the like. The moisture level indicator 50 may also be powered by a power supply 46, which directs power to the actuator 44 through the controller 52.

A power-on switch 54 may be provided in communication with the controller 52 for preserving the life of the battery of the power supply 46. The power-on switch 54 may also be provided in series with the power supply 46. The controller 52 may include various programs in accordance with the present invention. For example, upon actuation of the power on switch 54, the controller 52 may drive the actuator 44 to indicate to the user that the moisture level indicator 50 is operational. The power-on indication may vary in range, amplitude, or pattern from that of the moisture level indication Additionally, the controller 52 may include a timer for driving the actuator 44 in incremental periods to let the user know that the moisture level indicator 50 is still operational. These operations may relieve anxieties of the user associated with the concerns of the moisture absorbent sanitary apparatus reaching a full moisture level by informing the user that the moisture level indicator 26 is operational.

The moisture level indicator 50 may also include a speaker 56 for providing an audible alarm. The speaker 56 may chime when the power-on switch 54 is depressed to alert the user that the moisture level indicator 50 is in the moisture detection mode. Additionally, the controller 52 may convey the audible alarm from the speaker 56 in response to a signal from the moisture sensor 42 for alerting someone externally, other than the user, that the moisture absorbent sanitary apparatus has reached the predefined moisture level. This feature may be utilized for care of elderly, handicapped or infant persons.

The actuator 44, power supply 46, controller 52, power on switch 54, and speaker 56 may be provided within a cover 48 for preventing moisture from breaching these components and for ensuring the safety of the user.

The actuator 44 may impart a vibration or oscillation to the user to indicate that a moisture level has been reached. Alternatively, the actuator 44 may impart an electrical signal, such as an electrical stimulation to the user for indicating that the predefined moisture level has been reached. With reference to FIG. 6, the actuator 44 may be a miniature DC motor 58 with an eccentric weight 60 such that current imparted to the motor 58 causes the eccentric weight 60 to revolve, thereby creating a vibration or oscillation.

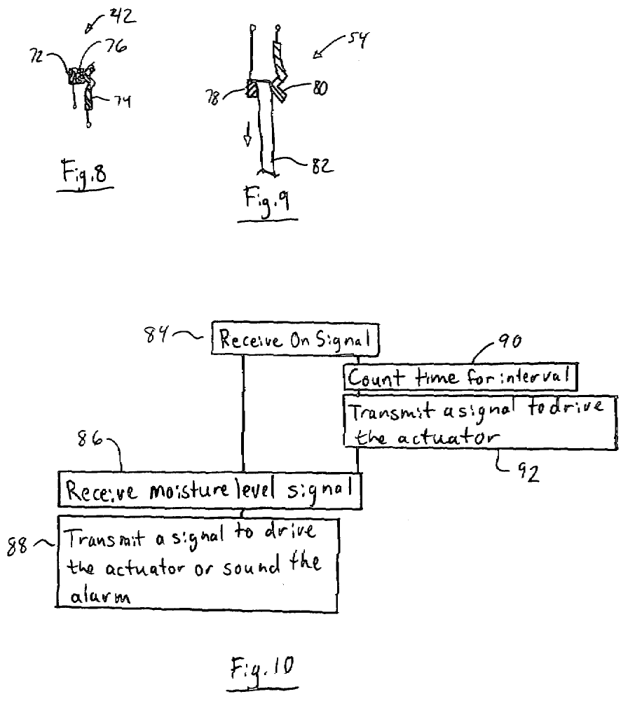

In FIG. 7, the actuator is illustrated schematically as a solenoid 62 that may be provided for imparting a vibration to the user. The solenoid 62 includes an inductive coil 64 located proximate to a magnet 66 whereby current imparted to the coil 64 imparts a dynamic motion to the magnet 66. The magnet 66 may be mechanically grounded by a spring 68 for resiliently supporting the magnet 66 and permitting continued motion after current is imparted to the coil 64. Further, capacitor 70 may be provided in series with the coil 64 for enhancing the acceleration of the initial motion imparted on the magnet 66. The solenoid 62 may be provided by a vibration chip or within an integrated circuit board for providing the vibration within a generally small volume.

The moisture sensor 42 may be a conventional moisture sensor. Alternatively, the moisture sensor 42 may be a normally-closed mechanical switch as illustrated in FIG. 8. The sensor 42 in FIG. 8 is illustrated with a fixed contact 72 and a flexible contact 74 biased into engagement with the fixed contact 72. A moisture soluble material 76, such as calcium, may be provided on the fixed contact 72 for preventing the switch from closing. Upon moisture reaching the moisture soluble material 76, the moisture soluble material 76 may dissolve thereby permitting the flexible contact 74 to translate into contact with the fixed contact 72 thereby closing the circuit.

The power-on switch 54 may be a manual switch that is depressed by the user. Alternatively, the power-on switch 54 may be a normally-closed switch having a fixed contact 78 and a flexible contact 80 that is loaded to be urged into engagement with the flexible contact 78. An insulating material such as a polymeric or plastic tab 82 may be provided between the flexible contact 80 and the fixed contact 78. The plastic tab 82 may extend out of the body of the moisture absorbent sanitary apparatus. When the user desires to power on the moisture level indicator 50, the user may remove the tab 82 from the sanitary apparatus.

With reference now to FIG. 10, a flowchart illustrates an exemplary method performed by computer-executable instructions of the controller 52. These instructions are not limiting, but merely illustrate a method and instructions for the method that may be provided within the controller 52.

Beginning with block 84, the controller includes instructions for receiving an ‘on’ signal. The on signal indicates that a moisture detection mode may begin. Therefore, the power supply 46 may be preserved until the ‘on’ signal is received.

Subsequently, in block 86, the controller 52 may include instructions for receiving a moisture level signal. Upon receipt of the moisture level signal, the controller 52 may include instructions in block 88 for transmitting a signal to drive the actuator 44 or sound the speaker 56.

The controller 52 may also include instructions for counting a time for an interval in block 90 after the ‘on’ signal is received in block 84. Once the time interval has lapsed, the controller 52 may include instructions for transmitting a signal to drive the actuator 44 as illustrated in block 92. Thus, the actuator 44 may periodically vibrate to indicate to the user that the moisture level indicator 50 is in operation. The interval may begin with a time of zero for initial indication of the power-on mode. Further, blocks 90 and 92 may be repeated for repetitive indication to the user that the moisture level indicator 50 is in operation.

In summary, a sanitary apparatus is provided with a moisture level indicator that discreetly and inconspicuously informs the user, or informs external persons in a simplified fashion that the sanitary apparatus has absorbed moisture to a certain level. Thus, anxiety and inconveniences associated with sanitary devices may be eliminated by providing certainty to the user.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A moisture absorbent sanitary apparatus comprising:
a body sized to cooperate with a body part of a user;
a moisture absorbent material provided in the body of the apparatus for absorbing moisture from the user;
a moisture sensor provided in the body of the apparatus for sensing moisture at a location of the body of the apparatus; and
an actuator provided in the body of the apparatus in communication with the moisture sensor for receiving a signal from the moisture sensor indicating a level of moisture absorption in the moisture absorbent material whereby the actuator imparts a signal to the body part of the user indicating to the user the level of moisture absorption.

2. The sanitary apparatus of claim 1 wherein the moisture sensor provides a signal to the actuator when the level of moisture absorption reached saturation of the moisture absorbent material.

3. The sanitary apparatus of claim 1 wherein the actuator further comprises a vibration mechanism for imparting a vibration to the body part of the user.

4. The sanitary apparatus of claim 1 wherein the actuator further comprises a vibration chip for imparting a vibration to the body part of the user.

5. The sanitary apparatus of claim 1 wherein the actuator is encased in a moisture resistant material.

6. The sanitary apparatus of claim 1 wherein the sensor is oriented within the body of the apparatus at a location to measure a partially full condition of the absorbent material.

7. The sanitary apparatus of claim 1 wherein the moisture sensor further comprises a switch that is adapted to be actuated by moisture.

8. The sanitary apparatus of claim 1 wherein the moisture sensor further comprises a normally-closed mechanical switch with a moisture soluble material maintaining the switch in a closed position until contacted by moisture.

9. The sanitary apparatus of claim 1 wherein the sanitary apparatus body is further defined as a tampon.

10. The sanitary apparatus of claim 1 wherein the sanitary apparatus body is further defined as a pad.

11. The sanitary apparatus of claim 1 wherein the sanitary apparatus body is further defined as a diaper.

12. The sanitary apparatus of claim 1 wherein the actuator further comprises an audible alarm.

13. The sanitary apparatus of claim 1 further comprising a power source.

14. The sanitary apparatus of claim 13 further comprising a manual switch for selecting a moisture detection mode.

15. The sanitary apparatus of claim 13 further comprising:
a normally-closed switch interrupting a circuit that includes the power source and the actuator; and
an insulator in cooperation with the switch maintaining the switch in an open orientation, wherein the user can remove the insulator for powering the circuit.

16. A moisture absorbent sanitary apparatus comprising:
a body sized to cooperate with a body part of a user;
a moisture absorbent material provided in the body of the apparatus for absorbing moisture from the user;
a moisture sensor provided in the body of the apparatus for sensing moisture at a location of the body of the apparatus; and
a vibration mechanism provided in the body of the apparatus in communication with the moisture sensor for receiving a signal from the moisture sensor indicating a level of moisture absorption in the moisture absorbent material whereby the vibration mechanism imparts a vibration to the body part of the user indicating to the user the level of moisture absorption.

17. A computer-readable medium having computer-executable instructions for performing a method comprising:
receiving a signal indicating a predefined moisture level in a moisture absorbent sanitary apparatus; and
transmitting a signal for driving an actuator in the moisture absorbent sanitary apparatus in response to receiving the signal for indicating to a user that the moisture absorbent sanitary apparatus has reached the predefined moisture level.

18. The computer-readable medium of claim 17 having computer-executable instructions for performing a method further comprising:
receiving a signal indicating commencement of a moisture detection mode before transmitting the signal to drive the actuator.

19. The computer-readable medium of claim 17 having computer-executable instructions for performing a method further comprising:
counting a time associated with predefined intervals; and
transmitting a signal after each interval for driving an actuator in the moisture absorbent sanitary apparatus for indicating to a user that a moisture sensor and the actuator of the moisture absorbent sanitary apparatus are operational.

20. The computer-readable medium of claim 17 having computer-executable instructions for performing a method further comprising:
transmitting a signal for emitting an audible alarm from the moisture absorbent sanitary apparatus in response to receiving the signal for indicating to a listener that the moisture absorbent sanitary apparatus has reached the predefined moisture level.
